(19) United States
(12) Patent Application Publication
(10) Pub. No.: US 2012/0082717 A1
(43) Pub. Date: Apr. 5, 2012

(54) COMPLEX, MULTILAYER USING THE SAME, AND DEVICE COATED WITH THE MULTILAYER

(75) Inventors: Kook Heon Char, Seoul (KR); Sai Bom Park, Seoul (KR); Jin Hwa Seo, Seoul (KR); Sang Beom Kim, Daejeon (KR); Chong Su Seo, Seoul (KR)

(73) Assignees: SNU R&DB FOUNDATION, Seoul (KR); ILJIN MATERIALS CO., LTD., Iksan (KR)

(21) Appl. No.: 13/376,143

(22) PCT Filed: Jun. 4, 2010
(86) PCT No.: PCT/KR2010/003614

§ 371 Date: Dec. 2, 2011

(30) Foreign Application Priority Data
Jun. 5, 2009 (KR) 10-2009-0050027
Jan. 21, 2010 (KR) 10-2010-0005769

(51) Int. Cl. A61K 9/127; A61K 31/337; C08B 37/08; A61K 31/7088; A61K 38/02; A61K 38/44; A61K 38/06; A61K 31/7076; A61K 31/7048; A61K 31/7028; A61K 9/00; A61P 35/00; A61P 7/02; A61P 31/04; A61P 29/00; A61P 5/24; A61P 37/06; A61P 31/12; A61P 23/00; A61P 1/08; A61P 37/08; B32B 27/08; B32B 9/04; B32B 27/06; B32B 5/00; A61L 27/54; A61L 27/50; A61F 2/00; A61K 31/59; B82Y 5/00

(52) U.S. Cl. 424/450; 514/167; 514/449; 536/55.1; 514/44 R; 514/1.1; 424/94.4; 514/21.9; 514/46; 514/27; 514/25; 424/400; 428/403; 428/532; 428/220; 428/474.4; 428/480; 427/2.24; 623/1.1; 977/773; 977/906

(57) ABSTRACT

Disclosed herein is a complex, wherein micelles and/or liposomes dispersed in hyaluronic acids and/or hyaluronic acid derivatives, with a drug and/or functional material loaded in the micelles and/or the liposomes. The complex can release the drug and/or functional material in a controlled manner. Also, a multilayer using the complex, and a device coated with the multilayer are disclosed herein.

### TECHNICAL FIELD

The present invention relates to a complex, a multilayer using the complex, and a device coated with the multilayer.

### BACKGROUND ART

Metals are an important material in the biological, biomedical, and other fields. For example, there are medical devices which are designed to be used inside the body and supports for cell cultures which are made of metallic materials. Stents are among such medical devices. For example, a stent is an indispensible device for percutaneous coronary intervention in which the stent is used to prevent disease-induced angiostenosis.

Percutaneous coronary intervention is now recognized as the most effective therapy for cardiovascular diseases such as myocardial infarction, angina pectoris, coronary stenosis, etc. In percutaneous coronary intervention, a guidewire and a balloon catheter are introduced through the artery in the leg or the arm to the coronary artery and located in place, and then the balloon is inflated to keep the narrowed site open. This operation may be completely done simply by expanding the vessel wall with the aid of a balloon catheter. In most cases (approximately 70%), however, a stent which is a thin metal mesh is additionally inserted into the vessel.

Typically, stents are designed to support the expanded vessel wall. Stents made of metallic materials may be rejected by the body. In addition, metallic stents, after being located in place, press the blood vessel wall, causing the tissue cells of the vessel wall to undergo barotraumas. Often, barotraumas caused by the employment of a stent induce cells to rapidly proliferate and excessively cover up the stent, resulting in restenosis.

Cell culture supports adapted to provide environments in which cells grow in an adherent manner are required to continuously supply growth factors for cells that are difficult to culture and allow cells to easily adhere thereto.

In order to solve the above-described problems, studies have been focusing on materials applicable to the surface of metallic stents and methods used to apply a coat of the materials. Particular attention has been paid to coating materials which can be contained therein and release drugs or growth factors in a sustained manner. Under intensive study are Drug Eluting Stents (DES) which contain restenosis-preventing drugs therein and elute the drugs. Among them are sirolimus-eluting stents from Johnson & Johnson and paclitaxel-eluting stents from Boston Scientific. However, these stents are disadvantageous in that they rapidly deplete drugs and employ just one drug. It is therefore needed to control the release rate of drugs and to employ two or more drugs.

Meanwhile, the formation on a metal surface of a layer capable of eluting drugs or functional materials may be achieved by coating the metal with a proper material containing the drugs or functional materials. Conventionally, stent surfaces are modified with polymeric materials or various polymers are sequentially layered on stents. In the early 1990s, Decher et al. developed and published papers on the so-called ‘layer-by-layer (LbL) self-assembly method’ (G. Decher, J. D. Hong, and J. Schmitt; Buildup of ultrathin multilayer films by a self-assembly process: Consecutively alternating adsorption of anionic and cationic polyelectrolytes on charged surfaces.; Thin Solid Films; 1992; 210/211; 831-835). Taking advantage of various forces including electrostatic attraction, hydrogen bonds and covalent bonds between polymer electrolytes, the method allows a multilayer coating to be very stably formed on the surface and can build an ultrathin film irrespective of the size and shape of the substrate to be coated.

Based on the layer-by-layer (LbL) self-assembly method, multilayer structures which contain therein drugs and slowly elute the drugs are studied. David M. Lynn et al. reported a multilayer structure plasmid DNA and hydrolysable polyamine that are layered in an alternating manner on stainless steel intravascular stent using the layer-by-layer (LbL) self-assembly method (C. M. Jewell, J. Zhang, N. J. Fredin, M. R. Wolff, T. A. Hacker, and D. M. Lynn; Release of Plasmid DNA from Intravascular Stents Coated with Ultrathin Multilayered Polyelectrolyte Films.; Biomacromolecules; 2006; 7; 2483-2491).

Also, Venkatraman et al. developed a bilayer system in which a lactide glycolide copolymer (PLGA; poly-D,L-lactide-co-glycolic acid) contains drugs with a poly L-lactide layer serving as a support, reporting the results of the application of the bilayer system to drug-eluting stents (Wang X T, Venkatraman S S, Boey F Y C, Loo J S C, and Tan L P; Controlled release of sirolimus from a multilayered PLGA stent matrix; Biomaterials.; 2006; 27; 5588-5595).

The stents developed thus far can contain drugs to a considerable extent, but are problematic in that it is difficult to control the content and eluting rate of drugs because the drugs are contained, as they are, in the polymeric layers. Because drugs are not effectively contained in the polymer layers and are considerably wasted during the preparation of the stents, the production cost increases.

Meanwhile, the present inventors reported a multilayer coat in which charged micelles with drugs loaded therein and linear polymers are alternately deposited (Jinhwa Seo, Jodie L. Lutkenhaus, Junoh Kim, Paula T. Hammond, and Kookheon Char; Development of Surface Morphology in Multilayered Films Prepared by Layer-by-Layer Deposition Using Poly(acrylic acid) and Hydrophobically Modified Poly(ethylene oxide); Macromolecules; 2007; 40(11); 4028-4036). According to the report, micelles consisting only of poly(acrylic acid) (PAA) and hydrophobically modified poly(ethylene oxide) (HM-PEO) block copolymers with functional materials entrapped therein and can be formed into multilayers having nano-sized pores. However, the polymer micelles, when used as they are, are difficult to control in terms of drug eluting rate and stable coat formation.

In order to overcome the problems encountered in the prior art, a multilayer structure, prepared using a layer-by-layer (Lbl) self-assembly method, must effectively contain drugs or functional materials therein. Further, the various parameters of the multilayer structure must be able to control the eluting rate and content of the drugs or functional materials. Particularly for stents, two types of drugs which are released at the early stage and over a long period of time are preferably used. Thus, a multilayer structure applied to stents must contain the two types of drugs therein at the same time and must be able to individually control the eluting rates of the drugs.

It is reported that when coated with polymer brush layers, metallic materials are improved in biocompatibility. As for polymer brush layers and methods of forming the same, representative references may be made to: (1) H. Ma, J. Hyun, P. Stiller, A. Chilkoti; “Non-Fouling” Oligo(ethylene glycol)—Functionalized Polymer Brushes Synthesized by Surface-Initiated Atom Transfer Radical Polymerization; Advanced Materials; 2004; 16(4); 338-341, (2) Jon Ladd, Zheng Zhang, Shengfu Chen, Jason C. Hower and Shaoyi Jiang; Zwitterionic Polymers Exhibiting High Resistance to Nonspecific Protein Adsorption from Human Serum and Plasma; Macromolecules; 2008; 9(5); 1357-1361, (3) Kazuhiko Ishihara, Runa Aragaki, Tomoko Ueda, Akihiko Watenabe, Nobuo Nakabayashi; Reduced thrombogenicity of polymers having phospholipid polar groups; Journal of Biomedical Materials Research; 1990; 24(8); 1069-1077, which are incorporated by reference in their entireties herein. A polymer brush layer can remain on a metallic material even if the other coating layers are degraded. Hence, it can be stably used in the body.

Leading to the present invention, intensive and thorough research into a multilayer with drugs or functional materials loaded therein, conducted by the present inventors, resulting in the finding that when used in combination with hyaluronic acid or a derivative thereof, micelles or liposomes with drugs or functional materials loaded therein can be deposited using a layer-by-layer (LbL) self-assembly process to form a multilayer which can release the drugs or functional materials in a controlled manner. Also, the multilayer can be applied to substrates useful in various fields. In addition, when intercalated between the multilayer and the substrate, a polymer brush layer guarantees the safe use of the substrate in the body even after the multilayer completely degrades.

### DISCLOSURE

### Technical Problem

An object of the present invention to provide a complex comprising micelles and/or liposomes, with drugs and/or functional materials loaded therein, which can effectively control the eluting rate of the drugs and functional materials and are dispersed in hyaluronic acids and/or hyaluronic acid derivatives, and a method for preparing the same.

Another object of the present invention is to provide a multilayer in which 1) i) a layer composed of the complex of the present invention, or ii) a layer composed of hyaluronic acids and/or hyaluronic acid derivatives; and 2) a layer composed of a positively charged polymer are formed alternately, and a method for preparing the same.

A further object of the present invention is to provide a device coated with the multilayer and a method for preparing the same.

### TECHNICAL SOLUTION

In accordance with an aspect thereof, the present invention provides a complex in which micelles and/or liposomes are dispersed in hyaluronic acids and/or hyaluronic acid derivatives.

As used herein, the term “micelle” refers to an aggregate of molecules having both hydrophilic and hydrophobic moieties. In an aqueous solvent, such as water, these molecules, also known as amphipathic molecules, form micelles in which the hydrophilic moieties are in contact with the surrounding solvent to generate a corona while sequestering the hydrophobic moieties in the micelle center to generate a core. When mixed with micelles, drugs and/or functional materials may be entrapped within the cores of the micelles. Hence, micelles can serve as storage or carriers for drugs or functional materials. Particularly, hydrophobic drugs or functional materials may be contained within the cores.

Preferably, the micelles range from 40 nm to 100 nm in mean size (diameter). For example, when they are larger than 100 nm, the micelles are unsuitable for delivering the drugs or functional materials because they are likely to be directed toward the kidney. On the other hand, micelles smaller than 40 nm are apt to undergo phagocytosis by macrophages, so that they cannot effectively deliver the drugs or functional materials. In addition, micelles with a mean size of from 40 nm to 100 nm can be stably dispersed in hyaluronic acid or hyaluronic acid derivative and can form a multilayer with a positively charged polymer electrolyte in a controllable manner with regard to the thickness of the multilayer. Preferably, the mean size of the micelles falls within the range of from 45 nm to 65 nm.

The micelles degrade with time, thus gradually releasing the drugs and/or functional materials entrapped within the cores thereof. The structure of a micelle according to an embodiment of the present invention is schematically illustrated in FIG. 1.

The amphipathic molecule serving as a constituent of the micelle is preferably a polymer. Polymers are suitable for controlling the size of micelles because the hydrophilic and the hydrophobic moieties are easy to deal with. Accordingly, it is easy to control the content of drugs and functional materials in the micelles consisting of polymers. The polymers may be mono- or copolymers having hydrophilic and hydrophobic moieties in their single molecules. When biocompatibility, degradation rate, and drug or functional material content are taken into account, the preferred polymer is one or more selected from PEG-PCL (polyethyleneglycol-polycaprolactone), PEG-PLA (polyethylene glycol-poly-L-lactic acid), PEG-PLL (polyethyleneglycol-poly-L-lysine), PEG-PEI (polyethyleneglycol-polyethylenimine), PEG-PLGA (polyethyleneglycol-poly(D,L-lactic-co-glycolic acid)), PEO-PPO (polyethylene oxide-polypropylene oxide) or PEG-PGA (polyethylene glycol-poly-glycolic acid).

Also, the ratio of the molecular weights of the co-monomers of the polymer has an influence on the sizes of the micelle core and corona and thus on the content of drugs or functional materials within the core. For example, when micelles are formed with PEG-PLGA, their core sizes can be adjusted with PEG_{2K}-PLGA_{2.32K} or PEG_{0.75K}-PLGA_{2.83K}, and thus the capacity of accommodating drugs and functional materials can be adjusted. The denotion “K” means a unit of molecular weight corresponding to 1000 daltons.

As used herein, the term “hyaluronic acid” refers to a compound consisting of repeating units composed of D-gluconic acid and N-acetyl glucose amine or to a salt thereof. Having the logarithmic measure of the acid dissociation constant, pKa, of about 2.9, hyaluronic acid, when dissolved in an aqueous solution, is dissociated into a negatively charged polymer electrolyte. The term “hyaluronic acid derivative”, as used herein, refers to a hyaluronic acid or its salt which is chemically modified at one or more functional groups thereof, and shows substantially the same functions as “hyaluronic acid”.

In order to be flexible enough to form networks through intermolecular and/or intramolecular bonds, the hyaluronic acid and/or its derivative preferably ranges in molecular weight from 300K to 2,000K. For example, hyaluronic acid with a molecular weight of 750K or 1,600K may be used.

As used herein, the term “dispersed” refers to a condition in which the micelles or the liposomes are independently accommodated by the networks formed by intermolecular and/or intramolecular hydrogen bonds of hyaluronic acid or derivatives thereof. As used herein, the term “network” refers to a space established by the hydroxyl or carboxyl groups of hyaluronic acid and/or derivatives thereof. Being similar in size to the micelle and/or the liposome, this space can stably accommodate the micelle and/or the liposome therein.

As used herein, the term “complex” refers to a structure in which micelles and/or liposomes are dispersed in and stably linked to hyaluronic acid and/or a derivative thereof. The complex according to an embodiment of the present invention is schematically illustrated in FIG. 2. Because the hyaluronic acid and/or the hyaluronic acid derivative of the complex is dissociated into negatively charged polymer electrolytes when dissolved in an aqueous solution, the complex can be used in layer-by-layer self-assembly deposition.

As used herein, the term “layer-by-layer self-assembly deposition” refers to a process by which positively and negatively charged polymer electrolytes are deposited in an alternating manner to yield a multilayer. By taking advantage of various interactions between polymer electrolytes, such as electrostatic interactions, hydrogen bonding, covalent bonding, etc., a multilayer can be stably formed on a substrate to be coated. When the complex of the present invention comprising micelles and/or liposomes with drugs and/or functional materials loaded therein is deposited thereinto using the layer-by-layer self-assembly process, the coated substrate, such as a medical device, can release the drugs or functional materials.

Further, the content of the micelles and/or the liposomes in the complex can be adjusted by varying the molar ratio between the micelles and/or the liposomes and the hyaluronic acids and/or hyaluronic acid derivatives, thereby determining the content and eluting rate of the drugs and functional materials. Preferably, the molar ratio between micelles and/or liposomes and hyaluronic acids and/or hyaluronic acid derivatives is in the range of from 1:10 to 10:1. In consideration of production efficiency, the molar ratio is preferably 1:3 to 1:7.

As described above, drugs or functional materials may be loaded within the core of the micelle and/or the liposome. The “drugs or functional materials” may be chosen in light of the various application fields.

As the drugs, sparingly water-soluble anticancer agents, e.g., having a solubility of less than 10 mg/mL, antithrombolic agents, anticoagulants, anti-bacterial agents, steroids, antiphlogistics, sex hormones, immunosuppressive agents, anti-viral agents, anesthetics, antiemetics, and anti-histamine agents may be used. Examples of the sparingly water-soluble drugs include paclitaxel, sirolimus, rapamycin, heparin, docetaxel, doxorubicin, cisplatin, carboplatin, 5-FU, etoposide, camptothecine, testosterone, estrogen, estradiol, triamcinolone acetonide, hydrocortisone, dexamethasone, prednisolone, betamethasone, cyclosporin and prostaglandin.

As the functional materials useful in the present invention, there are biocompatible materials such as DNA, RNA and polypeptides; skin whitening materials such as albutin, ethylascorbyl ether, ascorbyl glucoside, magnesium ascrobyl phosphate, ascorbic acid or derivatives thereof, kojic acid, glutathione, tyrosinase, diosmetin, macelignan, and vitamins or derivatives; anti-wrinkle agents, such as asiaticoside, ubidecarenone, polyethoxylated rethinamide, hydroxyproline, retinolic acid or derivatives thereof, alphahydroxic acid (AHA), adenosine, Botox or derivatives thereof; anti-inflammatory agents; anti-atopic agents; and anti-bacterial agents.

In accordance with another aspect thereof, the present invention provides a method for preparing a complex, comprising mixing micelles and/or liposomes with hyaluronic acids and/or hyaluronic acid derivatives. In an embodiment, a method for preparing a drug- or functional material-loaded complex, comprising mixing micelles and/or liposomes with a drug and/or a functional material to load drug- or functional material-entrapped micelles and/or liposomes; and mixing the drug-entrapped micelles and/or liposomes with hyaluronic acids and/or hyaluronic acid derivatives.

The complex and the method for preparing the same in accordance with the present invention are characterized as follows.

First, the micelle or the liposome of the complex according to the present invention may contain a drug or functional material in the core thereof. Such drug- or functional material-loaded micelles or liposomes are stably dispersed in hyaluronic acid or a hyaluronic acid derivative. Hyaluronic acid and derivatives thereof find applications in the medical, pharmaceutical and cosmetic fields. For example, they are used as a supplement for the joints, an aid for ophthalmic surgery, an orthopedic implant, a post-operative, adhesion barrier, or a filler for sustained release formulations or cosmetic formulations. In conjunction with the drug or functional material loaded, hyaluronic acid or derivatives thereof can be more effectively applied in these fields.

Second, because it comprises the negatively charged polymer electrolyte hyaluronic acid or hyaluronic acid derivative, the complex of the present invention can be used in layer-by-layer deposition. As a result, the multilayer thus formed contains the drugs or functional materials therein stably.

Third, the complex according to the present invention has various parameters affecting the content and eluting rate of the drug loaded therein. Among them are the micelle core with drugs or functional materials loaded therein, the molar ratio between the micelle (or the liposome) and the hyaluronic acid, and the molar concentration of the hyaluronic solution. By varying these parameters independently or in combination in consideration of the field to which the complex is applied, the content and eluting rate of the drugs or functional material can be readily controlled.

In accordance with a further aspect thereof, the present invention provides a multilayer in which a negatively charged layer and a positively charged layer are alternately formed, said negatively charged layer being composed of the complex of the present invention, or hyaluronic acids and/or hyaluronic acid derivatives, with the proviso that at least one negatively charged layer composed of the complex of the present invention must be present.

As used herein, the “multilayer” refers to that a negatively charged layer and a positively charged layer are stably deposited in an alternating manner using a layer-by-layer self-assembly process, with electrostatic attraction, hydrogen bonds and/or covalent bonds present therebetween. The structure of the multilayer is schematically illustrated in FIG. 3.

As used herein, the term “negatively charged layer” refers to a layer composed of the complex of the present invention, or hyaluronic acids and/or hyaluronic acid derivatives. Hyaluronic acids and/or derivatives thereof, serving as a main component of the negatively charged layer, has a logarithmic measure of the acid dissociation constant of about 2.9, and is dissociated into a negatively charged polymer electrolyte when dissolved in an aqueous solution so that it can be deposited using a layer-by-layer self-assembly process.

Due to the micelles and/or the liposomes contained therein, the layer composed of the complex according to the present invention can make a major contribution to the thickness of the multilayer. When drugs and/or functional materials are loaded to the micelles and/or the liposomes of the complex, the layer composed of the complex functions as a “reservoir of drugs or functional materials”.

A layer composed of the complex, but without drugs or functional materials loaded therein, or a layer composed of hyaluronic acid and/or a derivative thereof functions to control the eluting rate of the drugs or functional materials in the multilayer. An application of this multilayer is illustrated in FIG. 4. As seen in the diagram of FIG. 4, the multilayer comprises a rhodamine B-loaded layer and a coumarin 30-loaded layer with drug-free layers (spacer) intercalated therebetween. Being overlaid with no layers, the rhodamine B-loaded layer can release rhodamine B at the early stage. In contrast, coumarin 30 is released in a sustaining manner from the coumarin 30-loaded layer because a spacer is deposited thereon. This can be applied to a stent. In this regard, both of the two types of drugs one of which is required to be released just after the stent is implanted and the other over a long period of time, can be loaded onto one stent. In addition, the release time and eluting rate of the drug needed at the later stage can be controlled.

As used herein, the term “positively charged layer” refers to a layer composed of a positively charged polymer which can be useful in layer-by-layer deposition. The polymer is positively charged when it is dissociated in an aqueous solution. Considering that the positively charged polymer can be stably deposited through electrostatic attraction, hydrogen bonds and/or covalent bonds with hyaluronic acid, the positively charged polymer may be one or more selected from polyethyleneimine, poly-L-lysine, chitosan or polyaminoester.

With regard to the thickness of the multilayer, it is determined depending on the content and eluting rate of the drug or functional material loaded in the multilayer. Typically, the thickness can be adjusted with the number of the alternately deposited, positively and negatively charged layers. The multilayer is preferably at least 50 nm thick enough to contain sufficient amounts of drugs or functional amounts. In addition, the thickness of the multilayer preferably must not exceed 2 μm when production cost and the peeling of layers is taken into account.

As described above, the multilayer may contain drugs or functional materials therein. Two or more kinds of drugs or functional materials may be loaded in the same layer composed of the complex or respective layers composed of the complex. In the latter case, the drugs or the functional materials are released at different rates, which may be effective therapeutically.

In accordance with still a further aspect thereof, the present invention provides a method for preparing a multilayer, comprising 1) forming i) a layer comprised of the complex of the present invention, or ii) a layer comprised of hyaluronic acids and/or hyaluronic acid derivatives; 2) forming a layer composed of a positively charged polymer; and 3) repeating steps 1) and 2).

In accordance with still another aspect thereof, the present invention provides a device coated with the multilayer.

Preferably, the multilayer of the present invention is applied to devices which are designed to slowly release drugs or functional materials. Examples of such devices include artificial organs, such as stents, artificial hearts, heart-lung machines, ventricular assist devices; implantable sensors such as biochips; semiconductors; implantable medical devices such as bionic eyes; and supports for culturing cells. As used herein, the term “stent” refers to all kinds of stents and stent-like medical devices which are used to hold open the natural conduits formed to deliver fluids therethrough, such as blood vessels, digestive canals, intestinal tracts, gullets, bile ducts, bronchial tubes, etc.

The materials used in the devices may be selected depending on the application field to which the devices will be applied. Metals, such as stainless steel, tantalum, titanium, cobalt-chrome, nickel-titanium alloy (nitinol), and biodegradable magnesium-based alloy; alumina (Al₂O₃), which is biologically inert and maintains its morphology and structural integrity without causing an immunoreaction after implantation; zirconia (ZrO₂), which shows excellent mechanical properties; PMMA (Poly(methyl methacrylate)) bone cements; apatite bone cements; biologically active glass composed mainly of CaO and SiO₂ which can chemically bind to surrounding hard tissue; and biologically active ceramic consisting of the same ingredients as bone, such as calcium phosphate ceramic, are used as materials for the medical devices.

Preferably, the device has a polymer brush layer on the surface thereof, with the multilayer constructed on the polymer brush layer.

As used herein, the term “polymer brush layer” refers to a layer in direct contact with the surface of the device which is adapted to improve the coatability of the device with the multilayer and the biocompatibility of the device with biomaterials even after the multilayer has degraded.

The polymer brush layer may be formed of a polymer having at one terminus a functional group able to bond with the surface of the device, such as thiol, isocyanate, isothiocyanate or triethoxysilane, and at the other terminus a functional group able to bond with the multilayer, such as sulfur trioxide, carboxyl or phosphoryl choline. For example, OCN-PEO-SO₃H may be used.

Alternatively, the polymer brush layer may be formed of a polymer compound composed of 3-aminopropyl-triethoxysilane which bonds with the surface of the stent, and polyethylene glycol which has both an amino group and a carboxyl group reacting with the amino group of the 3-aminopropyl-triethoxysilane. The polyethyleneglycol having both carboxyl and amino groups preferably has a molecular weight of from 1 to 20 KDa.

A compound having sulfur trioxide ions may be linked to the side of the polymer compound which forms a bond with the drug reservoir layer (e.g., the amino group of polyethylene glycol). 1,1-dioxotetrahydrothiophene may be used as the compound having sulfur trioxide ions. Alternatively, sulfur trioxide ions may be linked to the hydrophilic polymer compound by polymerization (e.g., bulk polymerization) with a sulton compound, such as propane sulton.

The surface of the device may be preferably pre-treated so that it can form covalent bonds with or be crosslinked with the polymer brush layer. For example, after being introduced into the surface of the device from bis(5-carboxylpentyl)disulfide bis(pentafluorophenyl) ester, a pentafluorophenyl ester(PFP) group is reacted with a polymer having an amine group at its terminus to form a polymer brush layer.

Further, an additional coating material may be introduced into the pre-treated surface of the stent. An organic/inorganic hybrid polymer prepared by RAFT polymerization (Reversible Addition-Fragmentation Transfer Polymerization) may be suitable for use as the additional coating material. The organic/inorganic hybrid polymer is composed of an inorganic block based on silicon and is able to crosslink with the stent, and of an organic block which provides desired surface properties. Poly(silsesquioxanes) or APTES (Aminopropyltriethoxysilane) are suitable as the inorganic block. These inorganic blocks form covalent bonds with or are crosslinked with metal or metal oxide. The coated organic/inorganic hybrid polymer can be identified using contact angle measurement or silicon solid state NMR (nuclear magnetic resonance).

Even after the multilayer has worn out, the polymer brush layer, if present, prevents the direct contact of biomolecules with the device, guaranteeing the safe use of the device. Particularly, if given to the polymer brush layer at the region responsible for forming bonds with the multilayer, sulfur trioxide ions can semi-permanently prevent the adsorption of platelets and proteins onto the device because it functions as an anti-coagulant like hyrudin or heparin.

In accordance with yet another aspect thereof, the present invention provides a method for preparing a multilayer-coated device, comprising coating a device with a cationic polymer solution by immersion; washing the device; immersing the device in a dispersion of the complex of the present invention or a solution of hyaluronic acids and/or hyaluronic acid derivatives; washing the device; and optionally repeating the previous steps.

### Advantageous Effects

The complex, the multilayer, and the multilayer-coated device in accordance with the present invention exhibit the following features.

First, the multilayer can be formed using a layer-by-layer self-assembly process and is allowed by the complex to contain drugs or functional materials therein. A layer-by-layer self-assembly process can embody structurally stable multilayers irrespective of the size and morphology of the substrate, but is independent of the loading of drugs or functional materials into the multilayers. Thus, it is difficult to effectively control the content and eluting rate of drugs or functional materials. However, taking advantage of the fact that hyaluronic acid or derivatives thereof can stably disperse micelles therein, the present invention applies a layer-by-layer self-assembly process to the deposition of a dispersion of micelles or liposomes in hyaluronic acid so as to construct a multilayer with drugs or functional materials loaded therein, overcoming the problems encountered in the prior art.

Second, the complex and the multilayer according to the present invention are able to effectively control the content and eluting rate of the drugs or functional materials loaded therein. There are various factors that exert an influence on the content and eluting rate of the drugs or functional materials, including the kinds of the polymer components of the micelles or the liposomes, the molar ratio of micelles to hyaluronic acid or hyaluronic acid derivatives, and the concentration and molecular weight of hyaluronic acid or hyaluronic acid derivatives. Hence, the content and eluting rate of the drugs or functional materials can be controlled depending on the fields to which they are applied.

Third, the complex according to the present invention has advantages in terms of the economical aspect of drugs or functional materials. Conventional methods are not effective for loading drugs or functional materials to a polymer layer. Thus, a large quantity of drugs and functional materials are required due to the low yield, thereby increasing the production cost. In the present invention, drugs or functional materials are not directly loaded to a polymer layer, but are effectively entrapped within the micelles before incorporation into the layer. Therefore, drugs or functional materials can be contained at high yield without loss, thus decreasing the production cost.

### DESCRIPTION OF DRAWINGS

FIG. 1 is a schematic diagram illustrating the structure of a micelle with a drug loaded therein in accordance with an embodiment of the present invention.

FIG. 2 is a schematic diagram illustrating the structure of a polymer micelle-hyaluronic complex in accordance with an embodiment of the present invention.

FIG. 3 is a schematic diagram illustrating a multilayer in accordance with an embodiment of the present invention.

FIG. 4 is a schematic diagram illustrating a multilayer to which the polymer micelle-hyaluronic acid complex is applied in accordance with an embodiment of the present invention.

FIG. 5 is a graph of GPC chromatograms of the micelles in accordance with an embodiment of the present invention.

FIG. 6 is a graph of the size distribution determined by DLS of the micelles in accordance with an embodiment of the present invention.

FIG. 7 is a spectrum showing the content of the loaded drug comparative to that of the micelle in accordance with an embodiment of the present invention. FIG. 8 is a graph of the size distribution determined by DLS of the liposomes in accordance with an embodiment of the present invention.

FIG. 9 is a graph showing the thicknesses of the multilayers in accordance with an embodiment of the present invention plotted against the number of bilayers.

FIG. 10 is a set of atomic force microphotographs showing the surface morphologies of the multilayers in accordance with an embodiment of the present invention.

FIG. 11 is a set of atomic force microphotographs showing the surface morphologies of the multilayers in accordance with an embodiment of the present invention.

FIG. 12 is a set of scanning electron microphotographs showing the surface morphologies of the multilayers in accordance with an embodiment of the present invention.

FIG. 13 is a graph showing the thicknesses of the multilayers in accordance with an embodiment of the present invention plotted against the number of bilayers.

FIG. 14 is a set of scanning electron microphotographs showing the surface morphologies of the multilayers in accordance with an embodiment of the present invention.

FIG. 15 is a set of fluorescence microscope results showing the surface morphologies of the multilayers in accordance with an embodiment of the present invention.

FIG. 16 is a graph showing the eluting rate of the multilayers in accordance with an embodiment of the present invention.

FIG. 17 is a graph showing the eluting rate of the multilayers containing two functional materials in accordance with an embodiment of the present invention.

FIG. 18 is a set of atomic force microphotographs showing the surface morphologies of the multilayers in accordance with an embodiment of the present invention.

FIG. 19 is fluorescence microscope result showing the surface morphologies of the multilayers in accordance with an embodiment of the present invention.

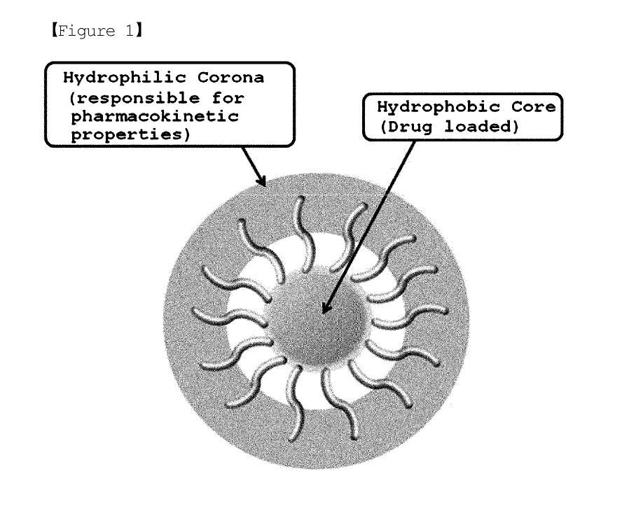

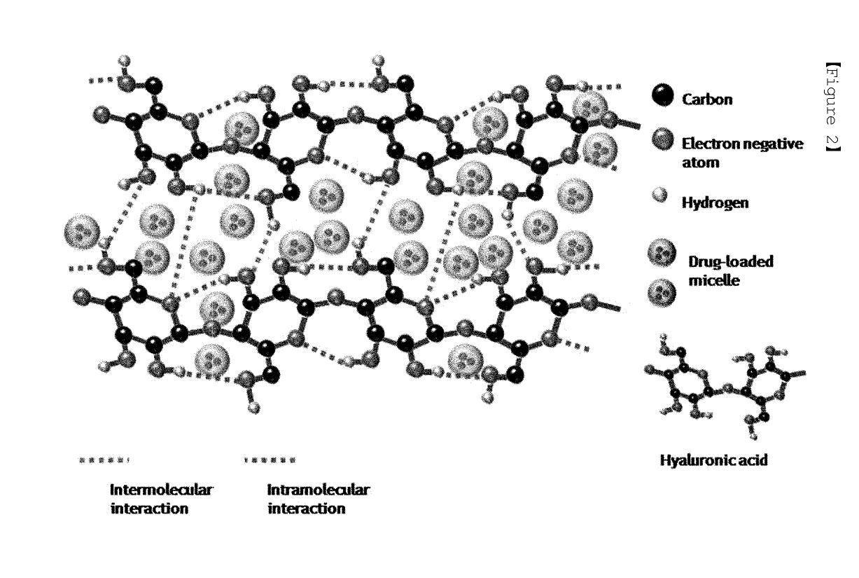

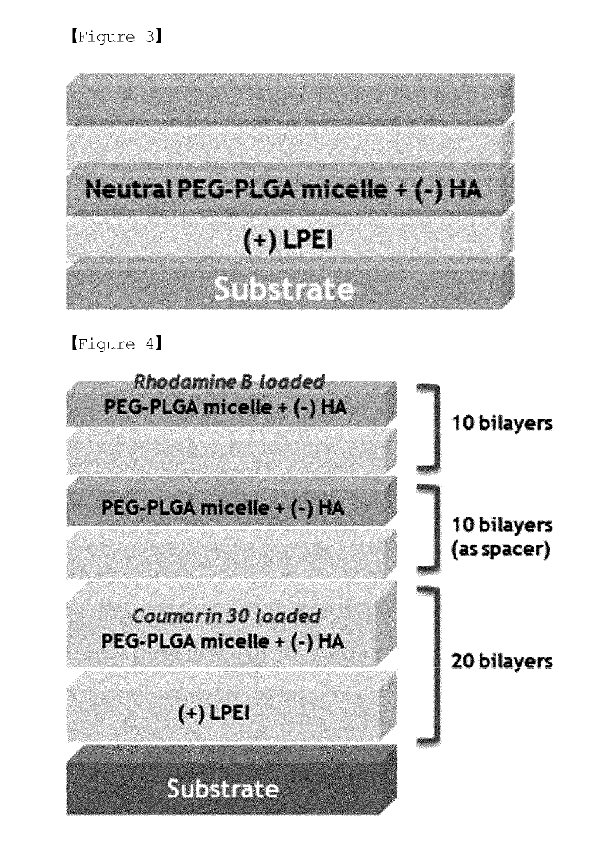

### MODE FOR INVENTION

A better understanding of the present invention may be obtained through the following examples which are set forth to illustrate, but are not to be construed as limiting the present invention.

### I. Preparation of Micelles and Liposomes

### EXAMPLE 1-1

### Preparation of Micelles

29.2 g of D,L-lactide, 2.6 g of glycolide, 18.2 g of monomethoxypolyethylene glycol (number mean molecular weight 2000), and 400 mL of toluene were placed in a nitrogen-filled round bottom flask and completely dissolved by heating at 120 for 1 hr. The molar ratio of D,L-lactide:glycolide was 9:1. Next, 0.32 grams of the reaction catalyst stannous octoate Sn(Oct)₂ was added to the round-bottom flask through a syringe, followed by polymerization at 120 for 24 hrs in a vacuum. The polymer thus formed was dissolved in methylene chloride and precipitated with diethyl ether. The precipitates were purified through filtration and dried in a vacuum.

The polymer was measured for a polydispersity value (1.2) using GPC and a glass transition temperature using a differential scanning calorimeter. H-NMR analysis showed that the polymer had a number average molecular weight of 4230 daltons. Monomethoxypolyethylene glycol-polylactide glycolide (mPEG-PLGA) was detected through FT-IR analysis. When PEG with different molecular weights or different amounts of D,L-lactide and glycolide are used to control the ratio of hydrophilic and hydrophobic groups, micelles with different properties, e.g., hairy or crew-cut type, could be obtained.

The compositions of the micelles prepared are given in Table 1, below.

TABLE 1

| Example 1-1 | Polymer | Molar Ratio between Polymers |
|---|---|---|
| Example 1-1 (1) | PEG, PLGA | PEG₂ₖ-PLGA_{2.32k} |
| Example 1-1 (2) | PEG, PLGA | PEG_{0.75k}-PLGA_{2.83k} |

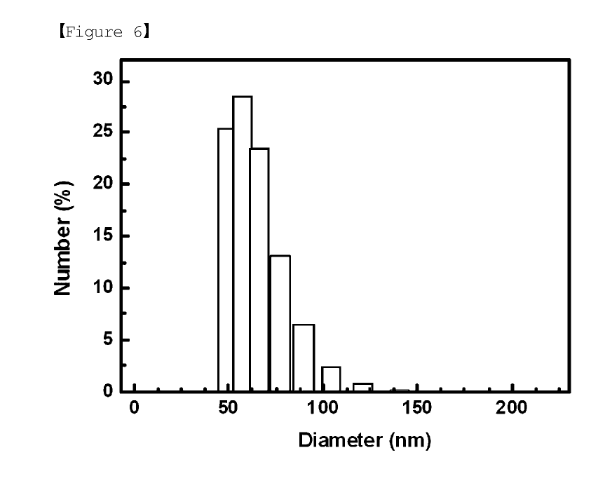

GPC chromatograms of the micelles are shown in FIG. 5.

### EXAMPLE 1-2

### Preparation of Micelles with Drugs or Functional Materials Loaded therein

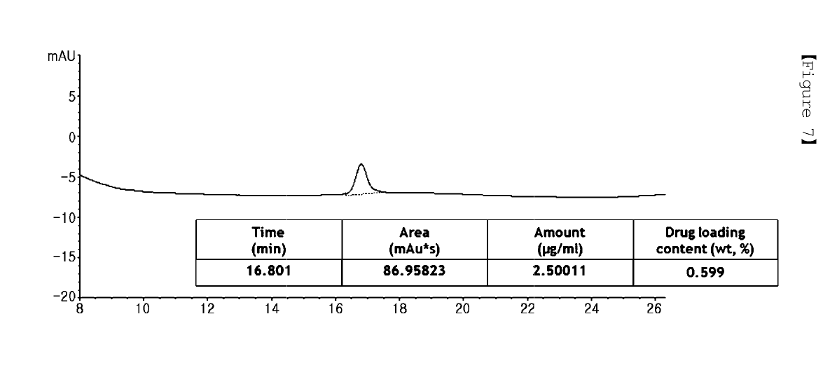

188 mg of the micelles (Example 1-1(1)) prepared in Preparation Example 1-1 was dissolved in 4 mL of N,N-dimethylformide to give a clear solution. At this time, the hydrophobic drug Paclitaxel, coumarin 30 or rhodamine B was added and introduced into the core of the micelle. The resulting solution was slowly added to 60 mL of distilled water while being stirred with a magnetic stirrer, followed by removing undissolved micelles through an organic solvent filter. The organic solvent was completely removed by stirring overnight at room temperature and then by dialysis for 1-2 days in a membrane. As a result, an aqueous solution in which micelles with drugs or functional materials loaded therein were dispersed was obtained. The size of the micelles was determined using dynamic light scattering (DLS), scanning electron microscopy, and atomic microscopy. The size distribution determined by DLS is shown in FIG. 6. As seen in FIG. 6, the micelles had a mean size (diameter) of 54.5 nm.

The compositions of the micelles containing Paclitaxel, coumarin 30 or rhodamine B therein are summarized in Table 2, below.

TABLE 2

| Example 1-2 | Micelle | Loaded Drug |
|---|---|---|
| Example 1-2 (1) | Example1-1 (1) | paclitaxel |
| Example 1-2 (2) | Example1-1 (1) | coumarin 30 |
| Example 1-2 (3) | Example1-1 (1) | rhodamine B |

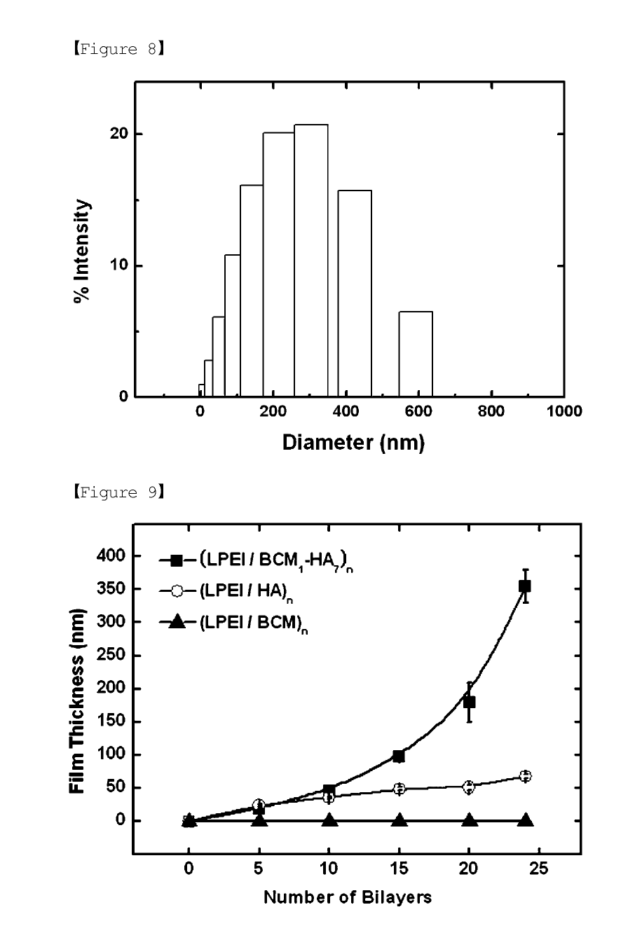

The micelles (PEO_{2K}-PLGA_{2.32K}) of Example 1-2(1) were measured for the content of paclitaxel using high performance liquid chromatography. On the basis of the fact that paclitaxel shows UV absorption peaks at 227 nm, the areas of the peaks detected at 16 min were used to calculate molecular weights of the drugs in comparison to those of the polymer micelles to determine the contents of the drugs loaded. The results are given in FIG. 7 and the drug loading content was approximately 0.599%.

### EXAMPLE 1-3

### Preparation of Liposomes

10 mL of chloroform was placed in a 250 mL flask having an opaque glass neck. 0.05 g of 1,2-Dioleoyl-sn-glycero-3-phosphocholine (MW: 786.15 g/mol), 0.04 g of cholesterol (95%), and 5 mg of 1,2-dihexadecanoyl-sn-glycero-3-phophoethanol amine (MW: 1333.81 g/mol) with rhodamine B attached, were added to the flask and completely dissolved by bath-type sonicator. The flask was fixed to a rotary evaporator, and the bottom part of the flask was immersed in 38° C. water. The temperature was kept until all the solvent in the flask was evaporated and lipid film was formed on the wall of the flask. In order to completely remove the remaining solvent, the flask was kept in a vacuum drier for one day. In order to hydrate the film, 20 mL water was added to the flask and the temperature was controlled to remain slightly high. Further, according to the structure of liposomes, the film was hydrated fully by using a bath-type or probe-type ultrasonicator, then liposome suspension was obtained by using a 0.45 μm filter for equalizing the size of liposomes.

The size of the liposomes was determined using dynamic light scattering (DLS). The size distribution determined by DLS is shown in FIG. 8.

### II. Preparation of Complex

### EXAMPLE 2

### Preparation of Complex

A 0.02 M micelle dispersion (Example 1-2(1)), prepared in Preparation Example 1-2, and a 0.02 M hyaluronic acid (1600K or 750K) solution were mixed at such ratios as to adjust the acidity to pH 3. Stirring with a magnetic stirrer produced complexes comprising the micelles which were dispersed in hyaluronic acid (Examples 2-1 to 2-8). Alternatively, when the molar ratio was fixed, different complexes were prepared using various concentrations of hyaluronic acid.

In addition, complexes were prepared in the same manner with exception that the micelle (Example 1-2(2)) prepared in Preparation Example 1-2 was employed (Examples 2-9 to 2-16).

In addition, a complex comprising the micelle (Example 1-2(3)) prepared in Preparation Example 1-2 was prepared (Example 2-17).

In addition, a complex containing neither drugs nor functional materials was prepared by mixing hyaluronic acid with the micelle (Example 1-1(1)) of Preparation Example 1-1 (Example 2-18).

In addition, a complex comprising the liposome prepared in Preparation Example 1-3 was prepared (Example 2-19).

The compositions of the prepared complexes are summarized in Table 3, below.

TABLE 3

| | | | Molar ratio | |
|---|---|---|---|---|
| | | | of Micelle | |
| | Mw of | | (or Liposome) | Drug or |
| | Hyaluronic | Micelle (or | to Hyaluronic | Functional |
| Example 2 | acid | Liposome) | acid | Material |
| Example 2-1 | 1600K | Example 1-2(1) | 1:7 | Paclitaxel |
| Example 2-2 | 1600K | Example 1-2(1) | 1:3 | Paclitaxel |
| Example 2-3 | 1600K | Example 1-2(1) | 3:1 | Paclitaxel |
| Example 2-4 | 1600K | Example 1-2(1) | 7:1 | Paclitaxel |
| Example 2-5 | 750K | Example 1-2(1) | 1:7 | Paclitaxel |
| Example 2-6 | 750K | Example 1-2(1) | 1:3 | Paclitaxel |
| Example 2-7 | 750K | Example 1-2(1) | 3:1 | Paclitaxel |
| Example 2-8 | 750K | Example 1-2(1) | 7:1 | Paclitaxel |
| Example 2-9 | 1600K | Example 1-2(2) | 1:7 | Coumarin 30 |
| Example 2-10 | 1600K | Example 1-2(2) | 1:3 | Coumarin 30 |
| Example 2-11 | 1600K | Example 1-2(2) | 3:1 | Coumarin 30 |
| Example 2-12 | 1600K | Example 1-2(2) | 7:1 | Coumarin 30 |
| Example 2-13 | 750K | Example 1-2(2) | 1:7 | Coumarin 30 |
| Example 2-14 | 750K | Example 1-2(2) | 1:3 | Coumarin 30 |
| Example 2-15 | 750K | Example 1-2(2) | 3:1 | Coumarin 30 |
| Example 2-16 | 750K | Example 1-2(2) | 7:1 | Coumarin 30 |
| Example 2-17 | 1600K | Example 1-2(3) | 1:7 | Rhodamine B |
| Example 2-18 | 1600K | Example 1-1(1) | 1:7 | (none) |
| Example 2-19 | 1600K | Example 1-3 | 1:7 | Rhodamine B |

### III. Preparation of a Substrate

### EXAMPLE 3-1

### Preparation of a Substrate Containing Paclitaxel

A layer-by-layer self-assembly process was performed in such a manner that a substrate was immersed in a 0.02 M solution of the cationic polymer LPEI (linear polyethyleneimine, Mw 25K), washed, immersed in a solution of the complex prepared in one of Examples 2-1 to 2-8 and washed again in that order to form a bilayer coat with paclitaxel loaded therein. This process was repeated until the number of the bilayer coat reached 5 to 25.

Analyses with an ellipsometer, a fluorescence microscope, a scanning electron microscope and an atomic force microscope confirmed that the cationic polymer layer and the anionic polymer layer were formed in an alternating manner.

### EXAMPLE 3-2

### Preparation of a Substrate Containing Coumarin 30

A layer-by-layer self-assembly process was performed in such a manner that a substrate was immersed in a 0.02 M solution of the cationic polymer LPEI (linear polyethyleneimine, Mw 25K), washed, immersed in a solution of the complex prepared in one of Examples 2-9 to 2-12 and washed again in that order to form a bilayer coat with coumarin 30 loaded therein. This process was repeated until the number of the bilayer coat reached 5 to 25.

Analyses with an ellipsometer, a fluorescence microscope, a scanning electron microscope and an atomic force microscope confirmed that the cationic polymer layer and the anionic polymer layer were formed in an alternating manner.

### EXAMPLE 3-3

### Preparation of a Substrate Containing Coumarin 30 and Rhodamine B

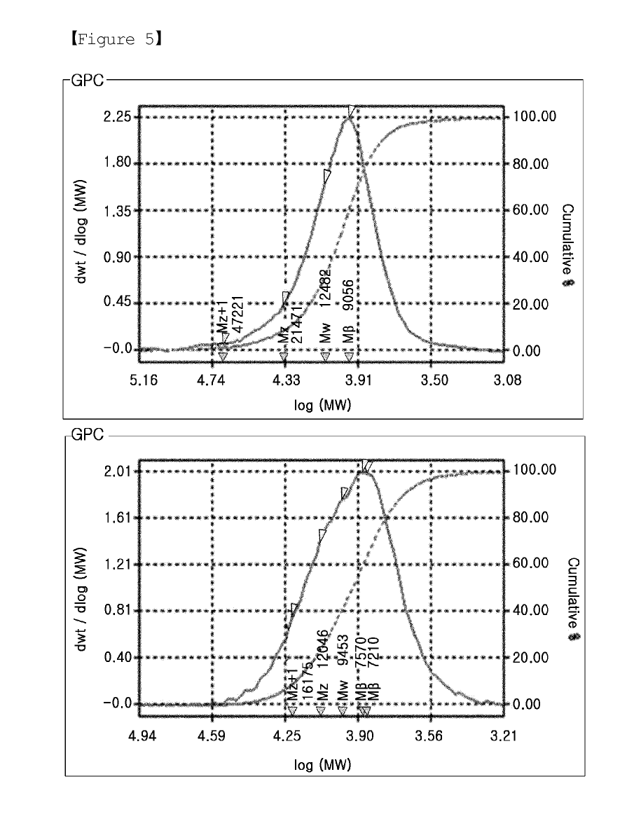

Multilayers were prepared in the same manner as in Example 3-1 with the exception that they were structured to contain coumarin 30 and rhodamine B in respective layers as in FIG. 4. The layer containing coumarin 30 was based on the complex of Example 2-9, the layer containing rhodamine B on the complex of Example 2-17, and the spacer on the complex of Example 2-18.

### EXAMPLE 3-4

### Preparation of a Substrate Containing Liposomes

A layer-by-layer self-assembly process was performed in such a manner that a substrate was immersed in a 0.02 M solution of the cationic polymer LPEI (linear polyethyleneimine, Mw 25K), washed, immersed in a solution of the complex prepared in Examples 2-19 and washed again in that order to form a bilayer coat with paclitaxel loaded therein. This process was repeated until the number of the bilayer coat reached 20.

Analyses with an ellipsometer, a fluorescence microscope, a scanning electron microscope and an atomic force microscope confirmed that the cationic polymer layer and the anionic polymer layer were formed in an alternating manner.

### COMPARATIVE EXAMPLE 1

### Preparation of a Substrate Non-Containing Micelles

The same procedure as in Example 3-1 was repeated, with the exception that hyaluronic acid (1600 K) free of micelles was used instead of the complex of the present invention.

### COMPARATIVE EXAMPLE 2

### Preparation of a Substrate by Using Only Micelles

The same procedure as in Example 3-1 was repeated, with the exception that only the micelle (Example 1-1(1)) was used instead of the complex of the present invention.

### IV. Properties of a Substrate

### EXPERIMENTAL EXAMPLE 1

### Measurement of Substrate for Thickness

The substrate of Example 3-1 and Comparative Examples 1 and 2 were measured for thickness using an ellipsometer and the results are given in FIG. 9.

As seen in the plot of FIG. 9, the multilayers of Example 3-1 (the complex of Example 2-1 employed) were observed to rapidly increase in thickness with the number of the bilayers. This was attributed to the fact that the micelle increased the surface roughness.

In contrast, when the hyaluronic acid free of micelles (LPEI/HA) was used as in Comparative Example 1, the multilayer was observed to very gradually increase with the number of the bilayer coat consisting of linear polyethylene imine and hyaluronic acid.

When only the micelle of Comparative Example 2 was used (LPEI/PEG-PLGA micelle) was employed, the thickness was not increased because no multilayers were formed.

### EXPERIMENTAL EXAMPLE 2

### Observation of Substrate Surface

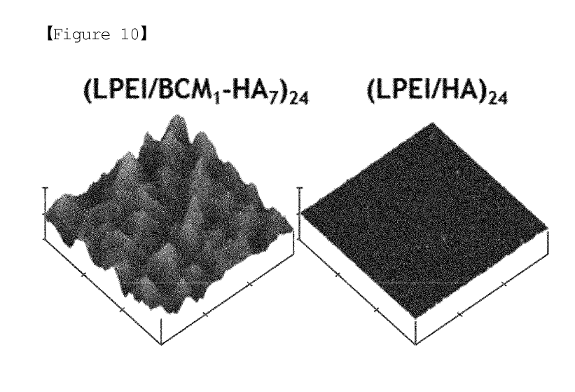

The substrate of Example 3-1 (employing the complex of Example 2-1) and Comparative Example 1 were observed under an atomic force microscope. The results are given in FIG. 10. As seen in FIG. 10, increases of surface roughness and thickness were found only in the substrate of Example 3-1.

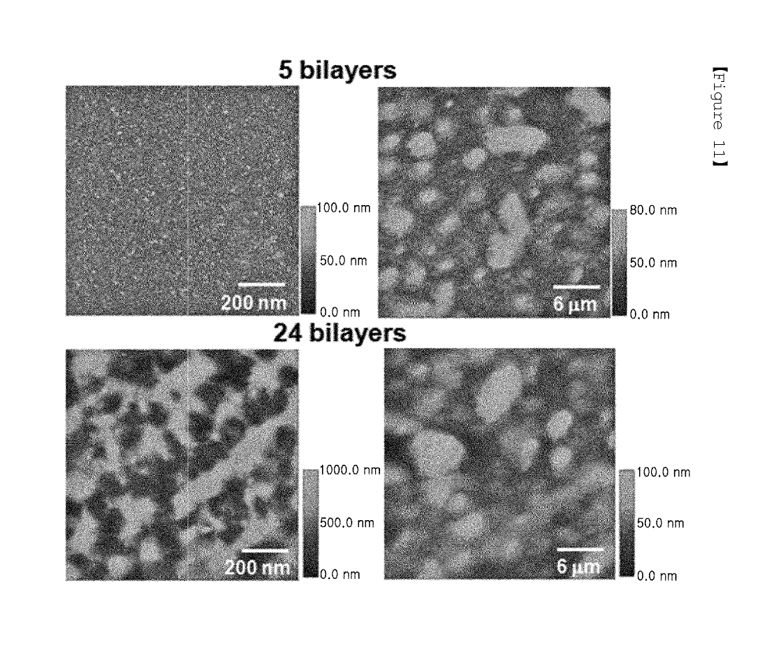

The substrates having 5 and 24 bilayer respectively, prepared in Example 3-1 (employing the complex of Example 2-1), were examined using an atomic force microscope, and the microphotographs are given in FIG. 11.

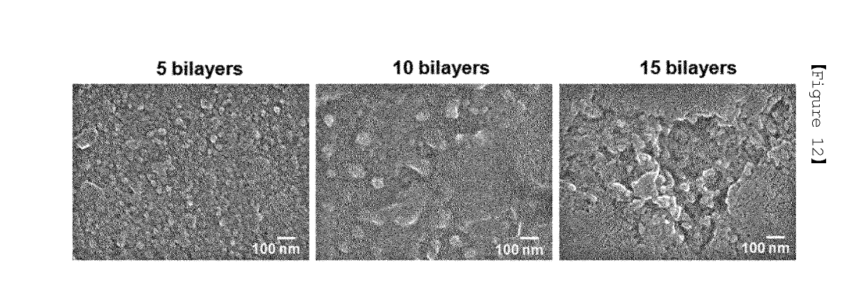

Also, the substrates having 5, 10 and 15 bilayer respectively, prepared in Example 3-1, were observed with a scanning electron microscope, and the results are given in FIG. 12.

As seen in FIGS. 11 and 12, the surface roughness was increased with the number of the bilayer coats while when a small number of the bilayer coats was deposited, micelles were comparatively evenly dispersed to form slightly rough surfaces.

### EXPERIMENTAL EXAMPLE 3

### Measurement of Coats for Thickness According to Molar Ratios

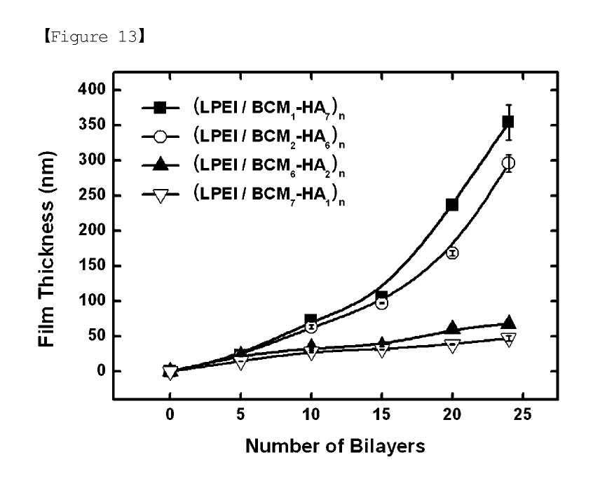

The multilayers formed in Example 3-1 (employing the complexes of Examples 2-1 to 2-4) were measured for thickness using an ellipsometer and the results are given in FIG. 13.

As seen in FIG. 13, all of the multilayers were observed to uniformly increase in thickness, but rapidly increase with increasing of the mole of hyaluronic acid.

### EXPERIMENTAL EXAMPLE 4

### Observation of Substrate Surface According to Molar Ratio

The multilayers prepared in Example 3-1 (employing complexes of Examples 2-1 to 2-4) were coated with platinum so as to prevent the multilayers from being electrically charged at surfaces thereof and from injured by strong electron light and to increase the discharge of secondary electrons.

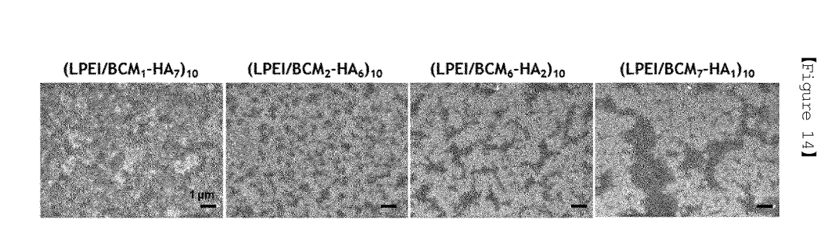

In this regard, a multilayer specimen was placed in a coating machine which was then vacuumed and purged with argon, after which platinum ions were injected to the surface of the multilayer specimen for 30 sec at 20 mA. The multilayer specimen thus plated with platinum was observed using a scanning electron microscope. The results are given in FIG. 14.

The dark part as seen in FIG. 14 indicates vacancy. As is apparent from the FIG. 14, micelles can be dispersed in hyaluronic acid effectively. Further, such effect is increased when molar ratio of hyaluronic acid is increased.

### EXPERIMENTAL EXAMPLE 5

### Observation of Substrate Surface According to Molar Ratio

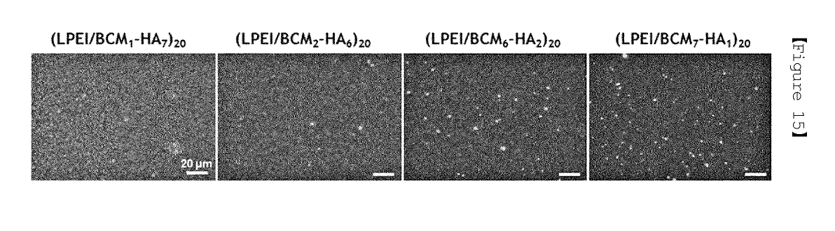

The content of coumarin 30 contained in the multilayers prepared in Example 3-2 (employing complexes of Examples 2-9 to 2-12) were observed using a fluorescence microscope. The results are given in FIG. 15.

As seen in FIG. 15, the higher intensity of fluorescence indicates that the more micelles are contained in the complex. Hence, by controlling the molar ratio of micelles to hyaluronic acid or hyaluronic acid derivatives, the content of the drugs or functional materials in the complex or multilayer can be controlled.

### EXPERIMENTAL EXAMPLE 6

### Measurement of Drug Eluting Rate

In a pH 7.4 buffered solution comprising sodium azide and Tween 20, a pH change induced the substrates of Exmaple 3-2 to degrade. Using a fluorescence analyzer, the fluorescent dye which was eluted from the multilayers during the degradation was quantitatively measured and the results are given in FIG. 16. As is apparent from the graph of FIG. 16, the drug eluting rate could be controlled according to the composition of the polymer complex.

### EXPERIMENTAL EXAMPLE 7

### Measurement of Drug Eluting Rates of Multilayers Containing Two or More Functional Materials therein

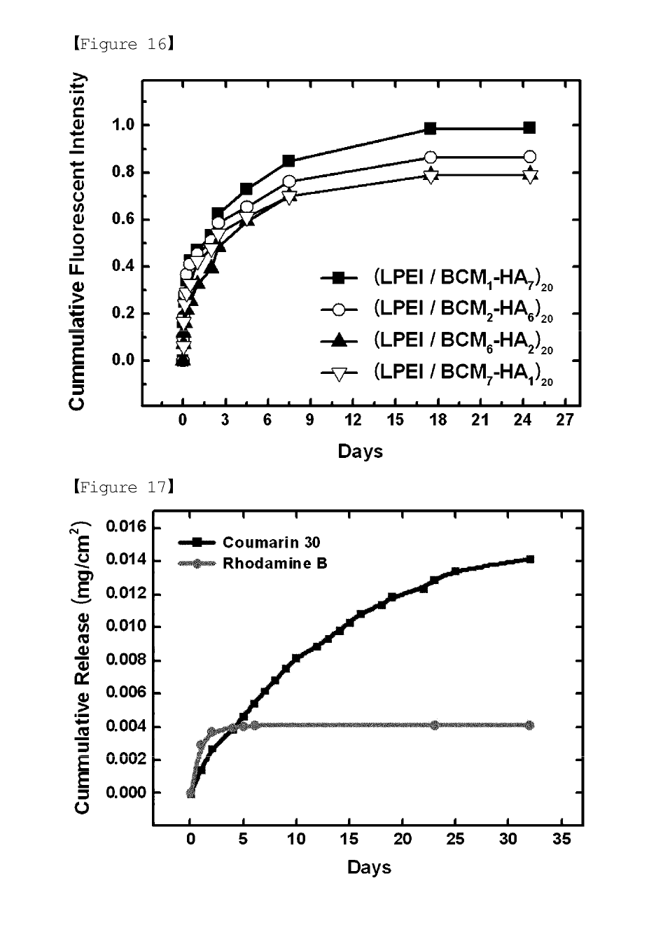

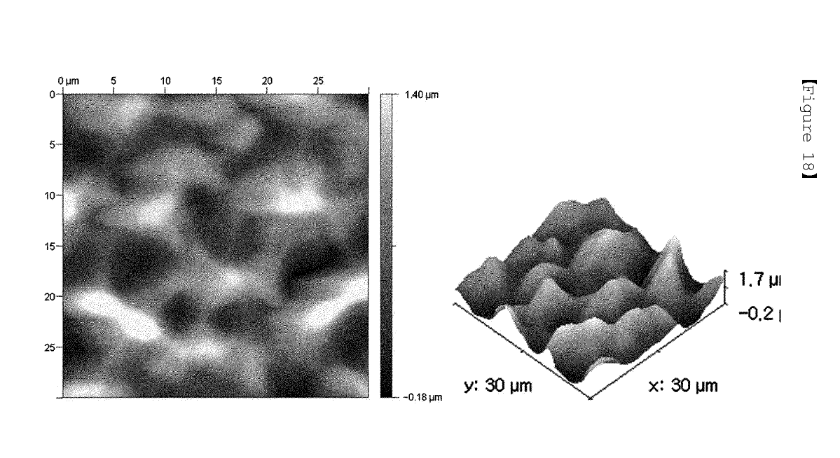

In a pH 7.4 buffered solution comprising sodium azide and Tween 20, a pH change induced the substrate of Exmaple 3-3 to degrade. Using a fluorescence analyzer, the functional materials which were eluted from the multilayers upon the degradation were quantitatively measured. The results are given in FIG. 17. As seen in the plot, rhodamine B was released at the early stage while coumarin 30 was slowly released over a long period of time.

### EXPERIMENTAL EXAMPLE 8

### Observation of Properties of a Substrate Containing Liposomes

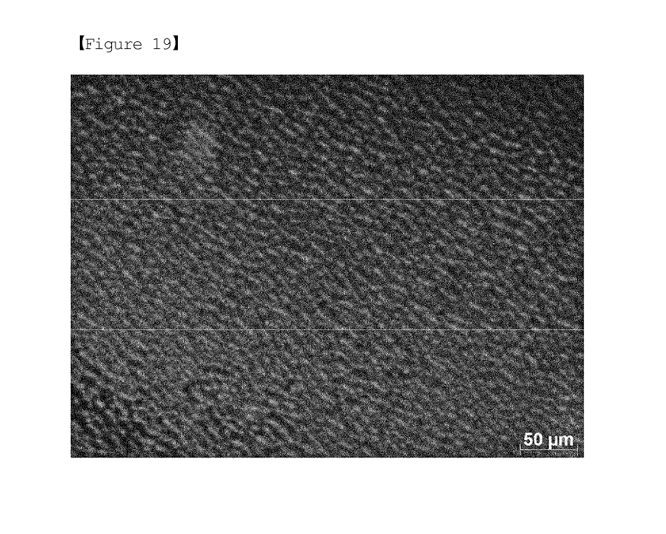

The substrate of Example 3-4 was observed under an atomic force microscope. The results are given in FIG. 18. As seen in FIG. 18, the surface roughness was observed as 381 nm.

Further, the intensity of fluorescence was observed using a fluorescence microscope. The results are given in FIG. 19. As seen in FIG. 19, not only micelles (about 50 nm diameter) but also liposomes (about 200 nm diameter), which have hydrophilicity and hydrophobicity, can be contained in hyaluronic acid.

What is claimed is:

1. A complex, wherein micelles and/or liposomes are dispersed in hyaluronic acids and/or hyaluronic acid derivatives.

2. The complex according to claim 1, wherein the micelles range in mean size of diameter from 40 nm to 120 nm.

3. The complex according to claim 2, wherein the micelles range in mean size of diameter from 45 nm to 65 nm.

4. The complex according to claim 1, wherein the micelles comprise one or more polymers selected from PEG-PCL(polyethyleneglycol-polycaprolactone), PEG-PLA(polyethylene glycol-poly-L-lactic acid), PEG-PLL(polyethyleneglycol-poly-L-lysine), PEG-PEI(polyethyleneglycol-polyethylenimine), PEG-PLGA(polyethyleneglycol-poly(D,L-lactic-co-glycolic acid)), PEO-PPO(polyethylene oxide-polypropylene oxide) or PEG-PGA(polyethylene glycol-poly-glycolic acid).

5. The complex according to claim 1, wherein the hyaluronic acids and/or the hyaluronic acid derivatives range in molecular weight from 300K to 2000K.

6. The complex according to claim 1, wherein the micelles and/or the liposomes are used at a molar ratio of from 1:10 to 10:1 to the hyaluronic acids and/or the hyaluronic acid derivatives.

7. The complex according to claim 6, wherein the micelles and/or the liposomes are used at a molar ratio of from 1:3 to 1:7 to the hyaluronic acids and/or the hyaluronic acid derivatives.

8. The complex according to claim 1, wherein the micelles and/or the liposomes contain a drug and/or a functional material therein.

9. The complex according to claim 8, wherein the drug is one or more selected from an anticancer agent, an antithrombolic agent, an anticoagulant, an anti-bacterial agent, a steroid, an antiphlogistic, a sex hormone, an immunosuppressive agent, an anti-viral agent, an anesthetic, an antiemetic or anti-histamine agent.

10. The complex according to claim 8, wherein the drug is one or more selected from paclitaxel, sirolimus, rapamycin, heparin, docetaxel, doxorubicin, cisplatin, carboplatin, 5-FU, etoposide, camptothecine, testosterone, estrogen, estradiol, triamcinolone acetonide, hydrocortisone, dexamethasone, prednisolone, betamethasone, cyclosporine or prostaglandin.

11. The complex according to claim 8, wherein the functional material is one or more selected from DNA, RNA, a polypeptide, albutin, ethylascorbyl ether, ascorbyl glucoside, magnesium ascrobyl phosphate, ascorbic acid or derivatives thereof, kojic acid, glutathione, tyrosinase, diosmetin, macelignan, vitamins or derivatives thereof, asiaticoside, ubidecarenone, polyethoxylated rethinamide, hydroxyproline, retinolic acid or derivatives thereof, alphahydroxic acid (AHA), Botox or derivatives thereof, or adenosine.

12. A multilayer, comprising:
1) i) a layer composed of the complex according to claim 1, or ii) a layer composed of hyaluronic acids and/or hyaluronic acid derivatives; and
2) a layer composed of a positively charged polymer, in an alternating pattern.

13. The multilayer according to claim 12, wherein the layer composed of the complex contains a drug and/or a functional material therein.

14. The multilayer according to claim 12, ranging in thickness from 50 nm to 2 μm.

15. The multilayer according to claim 12, wherein the positively charged polymer is one or more selected from polyethyleneimine, poly-L-lysine, chitosan or polyaminoester.

16. The multilayer according to claim 12, containing two or more different drugs or functional materials therein.

17. The multilayer according to 16, wherein the two or more different drugs or functional materials are loaded in respective layers.

18. A device, coated with the multilayer of claim 12.

19. The device according to claim 18, wherein a polymer brush layer is deposited directly on a surface of the device and overlaid with the multilayer.

20. The device according to claim 19, wherein the polymer brush layer is formed of a polymer having at one terminus a functional group able to bond with the surface of the device, selected from thiol, isocyanate, isothiocyanate and triethoxysilane, and at the other terminus a functional group able to bond with the multilayer, selected from sulfur trioxide, carboxyl and phosphoryl choline.

21. The device according to claim 18, wherein the device is selected from a stent, an artificial heart, a heart-lung machine, a ventricular assist device, a biochip, a semiconductor, and a bionic eye.

22. A method for preparing the complex of claim 1, comprising mixing micelles and/or liposomes with hyaluronic acids and/or hyaluronic acid derivatives.

23. A method for preparing the complex of claim 8, comprising:
mixing micelles and/or liposomes with a drug and/or a functional material to load the drug and/or the functional material therein; and
mixing the drug- and/or functional material-loaded micelles and/or liposomes with hyaluronic acids and/or hyaluronic acid derivatives.

24. A method for preparing the multilayer of claim 12, comprising:
1) forming i) a layer comprised of the complex of claim 1, or ii) a layer comprised of hyaluronic acids and/or hyaluronic acid derivatives;
2) forming a layer composed of a positively charged polymer; and
3) repeating steps 1) and 2).

25. A method for preparing the device of claim 18, comprising:
1) immersing a device in a cationic polymer solution to coat the device with the polymer;
2) washing the device;
3) coating the device with a solution of the complex of one of claims 1 to 11 claim 1, or hyaluronic acids and/or hyaluronic acid derivatives by immersion;
4) washing the device; and
5) repeating the steps 1) to 4).
